(19) United States
(12) Patent Application Publication
(10) Pub. No.: US 2011/0178286 A1
(43) Pub. Date: Jul. 21, 2011

(54) CONSUMABLE ANALYTICAL PLASTICWARE COMPRISING HIGH-SOLUBILITY PLASTICS

(76) Inventors: Joseph G. Bessetti, Cottage Grove, WI (US); Cristopher Cowan, Fitchburg, WI (US); Steve Krueger, Deerfield, WI (US); Paraj V. Mandrekar, Oregon, WI (US)

(21) Appl. No.: 13/011,241

(22) Filed: Jan. 21, 2011

Related U.S. Application Data
(60) Provisional application No. 61/297,035, filed on Jan. 21, 2010.

(51) Int. Cl. C07H 21/00; C08G 63/183; C08G 6/00; C08F 220/44; C08G 69/26

(52) U.S. Cl. 536/25.4; 528/308.1; 528/270; 526/338; 528/335; 422/527

(57) ABSTRACT

Consumable analytical plasticware comprising polymers having solubility parameters of about 9.5 (cal/cm³)^{1/2} or greater are described herein. Also disclosed are methods of using the consumable analytical plasticware, for example, to increase the yields of biomolecules such as nucleic acids from biological samples.

### CROSS REFERENCE TO RELATED APPLICATIONS

This application claims priority to U.S. Provisional Application No. 61/297,035, filed on Jan. 21, 2010, which is incorporated herein by reference in its entirety.

### BACKGROUND

Consumable analytical plasticware is commonly used in analytical laboratories, medical laboratories, forensic laboratories, hospitals, universities, etc. for the collection, storage, preparation, and processing of biological samples. Such plasticware is often made from polypropylene. Plasticware that results in increased yields of biomolecules from these biological samples is highly desirable.

### SUMMARY OF THE INVENTION

The invention provides, among other things, a process for isolating nucleic acids from a sample, comprising contacting the sample with consumable analytical plasticware comprising a polymer having a solubility parameter of 9.5 (cal/cm³)^{1/2} or greater while isolating the nucleic acids from the sample. The polymer may have a solubility parameter of about 11.0 (cal/cm³)^{1/2} or greater. The plasticware may be a plunger, a cartridge, a pipette tip, a sample vial, a tube, a multi-well plate, a dish, a syringe, a spatula, a probe, a swab, a section of tubing, a filter, a filter basket, or another vessel or device. The polymer may comprise polyethylene terephthalate (PET), polybutylene terephthalate (PBT), polyoxymethylene, acrylonitrile butadiene styrene (ABS), vinyl acetate, polyvinyl chloride (PVC), polyamide (nylon), or acrylonitrile. The polymer may be a homopolymer or a copolymer. The process may additionally comprise contacting the sample with silica, cellulose, a particle, a resin, or a paramagnetic material. The process may additionally comprise contacting the sample with an ion-exchange resin or a size-exclusion resin. The process may additionally comprise contacting the sample with consumable analytical plasticware comprising a polymer having a solubility parameter less than 9.5 (cal/cm³)^{1/2} to drive the nucleic acids toward the consumable analytical plasticware comprising a polymer having a solubility parameter of 9.5 (cal/cm³)^{1/2} or greater. For example, the process may use a plunger comprising a polymer having a solubility parameter of 9.5 (cal/cm³)^{1/2} or greater in conjunction with a cartridge comprising a polymer having a solubility parameter less than 9.5 (cal/cm³)^{1/2}. The sample may be an aqueous sample.

The invention additionally provides, among other things, a process for isolating nucleic acids from a sample, comprising contacting the sample with consumable analytical plasticware comprising a polymer having a solubility parameter greater than the solubility parameter of polypropylene while isolating the nucleic acids from the sample. The plasticware may be a plunger, a cartridge, a pipette tip, a sample vial, a tube, a multi-well plate, a dish, a syringe, a spatula, a probe, a swab, a section of tubing, a filter, a filter basket, or another vessel or device. The polymer may comprise polyethylene terephthalate (PET), polybutylene terephthalate (PBT), polyoxymethylene, acrylonitrile butadiene styrene (ABS), vinyl acetate, polyvinyl chloride (PVC), polyamide (nylon), or acrylonitrile. The polymer may be a homopolymer or a copolymer. The process may additionally comprise contacting the sample with silica, cellulose, a particle, a resin, or a paramagnetic material. The process may additionally comprise contacting the sample with an ion-exchange resin or a size-exclusion resin. The process may additionally comprise contacting the sample with consumable analytical plasticware comprising having a solubility parameter less than, or equal to, the solubility parameter of polypropylene to drive the nucleic acids toward the consumable analytical plasticware having a solubility parameter greater than the solubility parameter of polypropylene. For example, the process may use a plunger comprising a polymer having a solubility parameter greater than the solubility parameter of polypropylene in conjunction with a cartridge comprising a polymer having a solubility parameter less than, or equal to, the solubility parameter of polypropylene. The sample may be an aqueous sample.

The invention provides, among other things, consumable analytical plasticware comprising a polymer having a solubility parameter of about 9.5 (cal/cm³)^{1/2} or greater. The polymer may have a solubility parameter of about 11.0 (cal/cm³)^{1/2} or greater. The plasticware may be a plunger, a cartridge, a pipette tip, a sample vial, a tube, a multi-well plate, a dish, a syringe, a spatula, a probe, a swab, a section of tubing, a filter, a filter basket, or another vessel or device. The plasticware may be used to manipulate particle-bound or surface-bound nucleic acids. The polymer may comprise polyethylene terephthalate (PET), polybutylene terephthalate (PBT), polyoxymethylene, acrylonitrile butadiene styrene (ABS), vinyl acetate, polyvinyl chloride (PVC), polyamide (nylon), or acrylonitrile. The polymer may be a homopolymer or a copolymer.

The invention additionally provides, among other things, consumable analytical plasticware comprising a polymer having a solubility parameter greater than the solubility parameter of polypropylene. The plasticware may be a plunger, a cartridge, a pipette tip, a sample vial, a tube, a multi-well plate, a dish, a syringe, a spatula, a probe, a swab, a section of tubing, a filter, a filter basket, or another vessel or device. The plasticware may be used to manipulate particle-bound or surface-bound nucleic acids. The polymer may comprise polyethylene terephthalate (PET), polybutylene terephthalate (PBT), polyoxymethylene, acrylonitrile butadiene styrene (ABS), vinyl acetate, polyvinyl chloride (PVC), polyamide (nylon), or acrylonitrile. The polymer may be a homopolymer or a copolymer.

The invention additionally provides, among other things, a plunger comprising a polymer having a solubility parameter of about 9.5 (cal/cm³)^{1/2} or greater. The polymer may have a solubility parameter of about 11.0 (cal/cm³)^{1/2} or greater. The plunger may comprise polyethylene terephthalate (PET), polybutylene terephthalate (PBT), polyoxymethylene, acrylonitrile butadiene styrene (ABS), vinyl acetate, polyvinyl chloride (PVC), polyamide (nylon), or acrylonitrile. The plunger may be less than about 20 mm long and less than about 5 mm wide.

The invention additionally provides, among other things, a plunger comprising a polymer having a solubility parameter greater than the solubility parameter of polypropylene. The plunger may comprise polyethylene terephthalate (PET), polybutylene terephthalate (PBT), polyoxymethylene, acrylonitrile butadiene styrene (ABS), vinyl acetate, polyvinyl chloride (PVC), polyamide (nylon), or acrylonitrile. The plunger may be less than about 20 mm long and less than about 5 mm wide.

The invention additionally provides, among other things, a kit comprising consumable analytical plasticware comprising a polymer having a solubility parameter of about 9.5 (cal/cm³)^{1/2} or greater. The polymer may have a solubility parameter of about 11.0 (cal/cm³)^{1/2} or greater. The plasticware may be a plunger, a cartridge, a pipette tip, a sample vial, a tube, a multi-well plate, a dish, a syringe, a spatula, a probe, a swab, a section of tubing, a filter, a filter basket, or another vessel or device within which or upon which particle-bound or surface-bound nucleic acids are manipulated. The polymer may comprise polyethylene terephthalate (PET), polybutylene terephthalate (PBT), polyoxymethylene, acrylonitrile butadiene styrene (ABS), vinyl acetate, polyvinyl chloride (PVC), polyamide (nylon), or acrylonitrile. The kit may further include consumable plasticware comprising polypropylene.

The invention additionally provides, among other things, a kit comprising consumable analytical plasticware comprising a polymer having a solubility parameter greater than the solubility parameter of polypropylene. The plasticware may be a plunger, a cartridge, a pipette tip, a sample vial, a tube, a multi-well plate, a dish, a syringe, a spatula, a probe, a swab, a section of tubing, a filter, a filter basket, or another vessel or device within which or upon which particle-bound or surface-bound nucleic acids are manipulated. The polymer may comprise polyethylene terephthalate (PET), polybutylene terephthalate (PBT), polyoxymethylene, acrylonitrile butadiene styrene (ABS), vinyl acetate, polyvinyl chloride (PVC), polyamide (nylon), or acrylonitrile. The kit may further include consumable plasticware comprising polypropylene.

The invention additionally provides, among other things, a method of increasing the yield of isolated nucleic acids in a process for isolating nucleic acids from a sample, comprising contacting the sample with consumable analytical plasticware comprising a polymer having a solubility parameter of 9.5 (cal/cm³)^{1/2} or greater while isolating the nucleic acids from the sample. The polymer may have a solubility parameter of about 11.0 (cal/cm³)^{1/2} or greater. The method may additionally comprise contacting the sample with silica, cellulose, a particle, a resin, or a paramagnetic material. The method may additionally comprise contacting the sample with an ion-exchange resin or a size-exclusion resin. The method may additionally comprise contacting the sample with consumable analytical plasticware comprising a polymer having a solubility parameter less than 9.5 (cal/cm³)^{1/2} to drive the nucleic acids toward the consumable analytical plasticware comprising a polymer having a solubility parameter of 9.5 (cal/cm³)^{1/2} or greater. For example, the invention may use a plunger comprising a polymer having a solubility parameter of 9.5 (cal/cm³)^{1/2} or greater in conjunction with a cartridge comprising a polymer having a solubility parameter less than 9.5 (cal/cm³)^{1/2}. The sample may be an aqueous sample.

The invention additionally provides, among other things, a method of increasing the yield of isolated nucleic acids in a process for isolating nucleic acids from a sample, comprising contacting the sample with consumable analytical plasticware comprising a polymer having a solubility parameter greater than the solubility parameter of polypropylene while isolating the nucleic acids from the sample. The method may additionally comprise contacting the sample with silica, cellulose, a particle, a resin, or a paramagnetic material. The method may additionally comprise contacting the sample with an ion-exchange resin or a size-exclusion resin. The method may additionally comprise contacting the sample with consumable analytical plasticware comprising a polymer having a solubility parameter less than, or equal to, the solubility parameter of polypropylene to drive the nucleic acids toward the polymer having a solubility parameter greater than the solubility parameter of polypropylene. For example, the invention may use a plunger comprising a polymer having a solubility parameter greater than the solubility parameter of polypropylene in conjunction with a cartridge comprising a polymer having a solubility parameter less than, or equal to, the solubility parameter of polypropylene. The sample may be an aqueous sample.

The invention additionally provides, among other things, a method of increasing the yield of isolated nucleic acids in a process for isolating nucleic acids from a sample, comprising contacting the sample with consumable analytical plasticware comprising a polymer having a solubility parameter of 9.5 (cal/cm³)^{1/2} or greater while isolating the nucleic acids from the sample. The method may additionally comprise contacting the sample with silica, cellulose, a particle, a resin, or a paramagnetic material. The method may additionally comprise contacting the sample with an ion-exchange resin or a size-exclusion resin. The method may additionally comprise contacting the sample with consumable analytical plasticware comprising a polymer having a solubility parameter less than 9.5 (cal/cm³)^{1/2} to drive the nucleic acids toward the polymer having a solubility parameter of 9.5 (cal/cm³)^{1/2} or greater. For example, the invention may use a plunger comprising a polymer having a solubility parameter of 9.5 (cal/cm³)^{1/2} or greater in conjunction with a cartridge comprising a polymer having a solubility parameter less than 9.5 (cal/cm³)^{1/2}. The sample may be an aqueous sample.

The invention additionally provides, among other things, a method of increasing the yield of isolated biological molecules in a process for isolating biological molecules from a sample, comprising contacting the sample with consumable analytical plasticware comprising a polymer having a solubility parameter of 9.5 (cal/cm³)^{1/2} or greater while isolating the biological molecules from the sample. The method may additionally comprise contacting the sample with silica, cellulose, a particle, a resin, or a paramagnetic material. The biological molecules may be selected from the group consisting of proteins, lipids, sugars, carbohydrates, and enzymes.

The invention additionally provides, among other things, a method of increasing the yield of isolated biological molecules in a process for isolating biological molecules from a sample, comprising contacting the sample with consumable analytical plasticware comprising a polymer having a solubility parameter of 9.5 (cal/cm³)^{1/2} or greater while isolating the biological molecules from the sample. The method may additionally comprise contacting the sample with silica, cellulose, a particle, a resin, or a paramagnetic material. The biological molecules may be selected from the group consisting of proteins, lipids, sugars, carbohydrates, and enzymes.

Other aspects of the invention will become apparent by consideration of the detailed description and accompanying drawings.

### BRIEF DESCRIPTION OF THE DRAWINGS

FIG. 1 shows an embodiment of a plunger according to the invention.

FIG. 2 shows an embodiment of a pipette tip according to the invention.

FIG. 3 shows an embodiment of a microcentrifuge tube according to the invention.

FIG. 4 shows an embodiment of a multiwell plate according to the invention.

FIG. 5 compares the DNA extraction efficiency of a series of polymers used to contact a sample.

FIG. 6 compares the DNA extraction efficiency of polymers used to contact a sample.

### DETAILED DESCRIPTION

The invention provides consumable analytical plasticware comprising a polymer having a solubility parameter of about 9.5 (cal/cm³)^{1/2} or greater. It has been found that using consumable analytical plasticware comprising a polymer having a solubility parameter of about 9.5 (cal/cm³)^{1/2} or greater for the isolation of nucleic acids from a sample results in a better yield of isolated nucleic acids than using consumable analytical plasticware comprising a polymer having a solubility parameter of less than about 9.5 (cal/cm³)^{1/2}. Additionally, the use of a polymer having a solubility parameter of about 11.0 (cal/cm³)^{1/2} or greater will, in some cases, result in an even better yield of isolated nucleic acids than using consumable analytical plasticware comprising a polymer having a solubility parameter greater than about 9.5 (cal/cm³)^{1/2} but less than about 11.0 (cal/cm³)^{1/2}.

The solubility parameter (δ) of a polymer is defined as the square root of the cohesive energy density, a value which arises from the Flory-Huggins theory of polymers. See, Heimenz and Lodge, Polymer Chemistry, 2d. Ed. pp. 254-280 (2007), incorporated herein by reference in its entirety. Because δ is a square root function, the unit are typically expressed in (cal/cm³)^{1/2}. The solubility parameter describes the miscibility of a particular polymer being dissolved by a particular solvent, where the solvents also have corresponding solubility parameters. The more similar a polymer and a solvent are, the more likely that they are miscible. Table 1 shows a representative number of solubility parameters for polymers and solvents, however Table 1 in no way limits the materials suitable for use in the invention.

TABLE 1
Exemplary solubility parameters for polymers and solvents

| Polymer | δ (cal/cm³)^{1/2} | Solvent | δ (cal/cm³)^{1/2} |
|---|---|---|---|
| Polyethylene | 7.9 | Diethyl ether | 7.4 |
| Polybutadiene | 8.3 | Carbon | 8.6 |
| Polystyrene | 9.1 | tetrachloride | |
| Polypropylene | 9.3 | Toluene | 8.9 |
| Polyoxymethylene | 9.9 | Acetone | 9.9 |
| Vinyl acetate | 10 | Pyridine | 10.7 |
| Polyethylene | 10.7 | DMSO | 12.0 |
| terephthalate | | Glycerol | 16.5 |
| Acrylonitrile | 12.5 | Water | 23.4 |
| Nylon 6,6 | 13.6 | | |

Polymers suitable for use with the invention need not be limited to polymers containing all of the same monomer (i.e., homopolymers), as co-polymers having solubility parameters greater than about 9.5 (cal/cm³)^{1/2} are also suitable for use with the invention. Co-polymers suitable for the invention may comprise random co-polymers, block co-polymers, tapered block co-polymers and co-polymers thereof. The solubility parameter of a co-polymer must typically be measured experimentally, and varies depending upon the proportion of co-monomers as well as the conditions under which the co-polymer is created. In some embodiments, acrylonitrile butadiene styrene (ABS) copolymers such as TERLUX®, sold by BASF (Ludwigshafen, Germany) or polyoxymethylene resin polymers such as DELRIN®, having a solubility parameter greater than about 9.5 (cal/cm³)^{1/2}, are suitable for use with the invention. In some instances, cross-linking may increase the solubility parameter of a polymer having a solubility parameter less than about 9.5 (cal/cm³)^{1/2}, so that it is suitable for use with the invention.

Consumable analytical plasticware is commonly used in analytical laboratories, medical laboratories, forensic laboratories, hospitals, universities, etc. for the collection, storage, preparation, and processing of biological samples. In some embodiments, nucleic acids, especially deoxyribonucleic acids (DNA) and ribonucleic acids (RNA), will be isolated from the samples using consumable analytical plasticware according to the invention. In some embodiments, biological molecules, including, but not limited to, proteins, lipids, sugars, carbohydrates, and enzymes will be isolated from the samples using consumable analytical plasticware according to the invention. Consumable analytical plasticware may be disposed of after a single use, or it may be disposed of after several uses, or it may be disposed of after several dozen uses, or it may be disposed of after several hundred uses.

The consumable analytical plasticware may include any consumable container, sample holder, sample preparation device or implement that is used in conjunction with the handling of biological samples, however the use of the plasticware need not be limited to the isolation of nucleic acids. The plasticware may also be used for the isolation of other biological molecules, including, but not limited to, proteins, lipids, sugars, carbohydrates, and enzymes. In some embodiments, the plasticware will be constructed entirely from a polymer having a solubility parameter of about 9.5 (cal/cm³)^{1/2} or greater. In other embodiments, the plasticware will be partially constructed from a polymer having a solubility parameter of about 9.5 (cal/cm³)^{1/2} or greater. Consumable analytical plasticware may include, but need not be limited to, plungers, cartridges, pipettes, pipette tips, sample vials, tubing, tubes, multi-well plates, dishes, syringes, spatulas, probes, tubing, filters, filter baskets, or other vessels or devices within which or upon which particle-bound or surface-bound nucleic acids are manipulated.

While not wishing to be bound by theory, it is supposed that polymers having solubility parameters greater than about 9.5 (cal/cm³)^{1/2} may attract biological molecules within an aqueous sample, allowing the biological molecules to be more efficiently isolated from the sample. Conversely, it is supposed that polymers having solubility parameters less than about 9.5 (cal/cm³)^{1/2} (e.g., polypropylene, polystyrene) may repel biological molecules within an aqueous sample. Accordingly, it may be advantageous to store isolated biological molecules in polymers having solubility parameters less than about 9.5 (cal/cm³)^{1/2}, but to use polymers having solubility parameters greater than about 9.5 (cal/cm³)^{1/2} during isolation processes. Furthermore, isolation protocols using combinations of polymers having solubility parameters greater than about 9.5 (cal/cm³)^{1/2}, as well as polymers having solubility parameters less than 9.5 (cal/cm³)^{1/2}, may be more efficient at isolating biological molecules from a sample. For example, it may be beneficial to isolate biological molecules using an ABS (TERLUX®) plunger and a polypropylene reagent cartridge in an automated nucleic acid separation instrument.

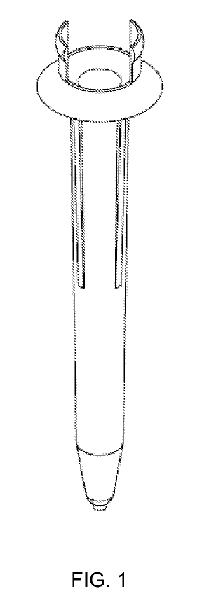

Plungers include devices which are used to stir, mix, shear, displace biological samples, or provide a surface upon which to attract, capture, hold, move or manipulate samples or components of samples. The samples may include nucleic acids or other biological molecules, including, but not limited to, proteins, lipids, sugars, carbohydrates, and enzymes. Plungers may be constructed in many shapes that are suitable for the processing of biological samples. In some embodiments, the plungers are substantially cylindrical and may have a closed end and an open end. Plungers according to the invention are less than about 50 mm in length, typically less than about 20 mm in length, more typically less than about 10 mm in length. Plungers according to the invention are less than about 20 mm wide, typically less than about 5 mm wide, more typically less than about 1 mm wide. In some embodiments, plungers of the invention are used for automated DNA isolation with robotics, such as using the MAXWELL® system (Promega Corporation, Madison, Wis.). Plungers of the invention may be used for manual isolation of DNA as well. Plungers of the invention may be made completely from polymers having a solubility parameter of greater than about 9.5 (cal/cm³)^{1/2}, however, plungers of the invention may include additional materials which do not have a solubility parameter of greater than about 9.5 (cal/cm³)^{1/2}. Plungers of the invention may have functionalized surfaces for more efficient isolation of nucleic acids. Plungers of the invention may also have particles bonded to the surface, such as, but not limited to, silica particles. An exemplary plunger is shown in FIG. 1.

Cartridges of the invention may be used to wash, collect, react, analyze, or isolate biological samples. Cartridges of the invention may also be used to store, prepare, dilute, or ship reagents, probes, nucleic acids, or particles for use in the isolation of nucleic acids. Cartridges of the invention may, for example, be used to prepare and ship sets of reagents and particles for use in automated DNA isolation with robotics, such as for the MAXWELL® system. Cartridges of the invention may have many different shapes, however in some embodiments, the cartridges of the invention will be substantially rectangular with a plurality of separated compartments for the separation of various reagents, etc.

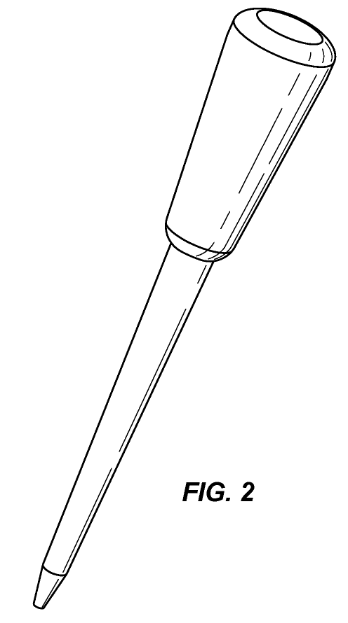

Pipette tips of the invention may be used to measure, extract, transport, dispense, or remove liquids. Pipette tips of the invention may be of any suitable volume, but typically are less than about 6 mL, typically less than about 300 μL, or less than about 50 μL. Pipette tips of the invention, comprising polymers having a solubility parameter of less than about 9.5 (cal/cm³)^{1/2}, may be of standard dimensions and volumes, to make them compatible with pipettes sold commercially, such as pipettes sold by Eppendorf (Hauppauge, N.Y.). Standard sizes include, but need not be limited to 10 μL, 20 μL, 200 μL, 1000 μL, and 5000 μL. Pipette tips of the invention may be constructed for specialized use or equipment, and need not be limited to standard sizes. An exemplary pipette tip is shown in FIG. 2. In one embodiment, pipette tips of the invention additionally comprise silica particles contacting an interior surface of the pipette tip. The invention additionally comprises plastic pipettes, such as 1, 5, 10, 25, 50, 100 mL plastic pipettes used for larger volume biological sample processing.

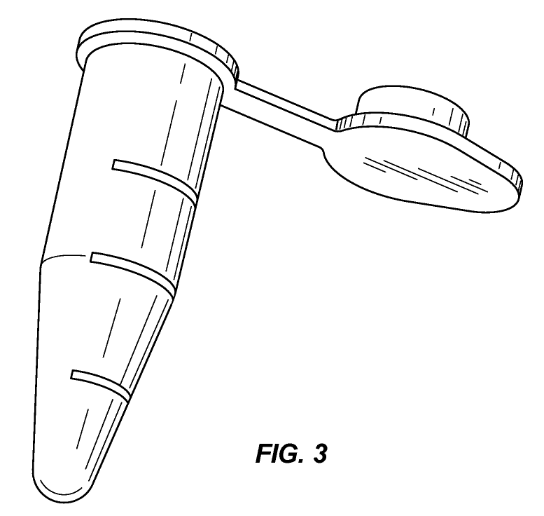

Tubes of the invention include test tubes, microcentrifuge tubes (microtubes), sample tubes, cuvettes, and conical tubes, (e.g., 15 and 50 mL conical tubes being commonly used for large volumes of biological materials). Tubes of the invention may be used to process, freeze, react, mix, or centrifuge liquids, solids, and mixtures of liquids and solids. Microcentrifuge tubes of the invention may be of any suitable volume, but typically are less than about 20 mL, typically less than about 10 mL, or less than about 2 mL. Microcentrifuge tubes of the invention, comprising polymers having a solubility parameter of more than about 9.5 (cal/cm³)^{1/2}, may be of a standard dimensions, to make them compatible with commercially available centrifuges, shakers, or trays. Microcentrifuge tubes are typically 1.5 mL in volume. An exemplary microcentrifuge tube is shown in FIG. 3.

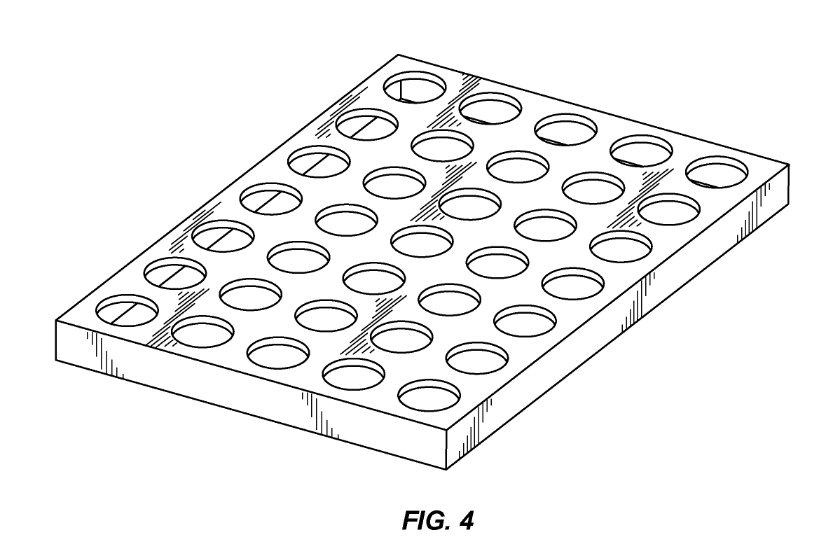

Multiwell plates of the invention may be used to react, observe, assay, mix, or prepare liquids, solids, and mixtures of liquids and solids. Multiwell plates have more than one well in a single article of consumable analytical plasticware. Multiwell plates have more than four, typically more than 20, more typically more than 80 wells for holding a liquid or a mixture of a liquid and a solid. Each well may have an identical volume, or the wells may have differing volumes. The volume of a well is less than 15 mL, more typically less than 5 mL, more typically less than 2.5 mL. The multiwell plates may have 24 wells, or 96 wells, or 384 wells. An exemplary multiwell plate is shown in FIG. 4.

The invention additionally includes kits that include consumable analytical plasticware comprising a polymer having a solubility parameter of about 9.5 (cal/cm³)^{1/2} or greater. The kits may additionally comprise reagents, particles, fluorescent markers, enzymes, and nucleic acids. The kits may be specialized for the isolation of nucleic acids from a biological sample and the amplification and quantization of the isolated nucleic acids, for example.

The invention additionally includes methods of increasing the yield of isolated nucleic acids in a process for isolating nucleic acids from a sample, comprising contacting the sample with consumable analytical plasticware comprising a polymer having a solubility parameter of 9.5 (cal/cm³)^{1/2} or greater while isolating the nucleic acids from the sample. Generally, the use of consumable analytical plasticware comprising a polymer having a solubility parameter of about 9.5 (cal/cm³)^{1/2} or greater has been shown to reduce the loss of nucleic acids in processes for isolating the nucleic acids from a sample. In one embodiment, the process of isolating nucleic acids involves preparation of a sample lysate followed by addition of a binding buffer and binding surface (particle or active surface) upon which the nucleic acids bind. The particle or surface bound nucleic acids are washed to remove components of the sample, lysis buffer, and binding buffer. Finally, the nucleic acids are recovered (eluted) into an appropriate storage buffer. However, the benefits of using consumable analytical plasticware comprising a polymer having a solubility parameter of about 9.5 (cal/cm³)^{1/2} or greater need not be limited to the isolation of nucleic acids from a sample.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any nonclaimed element as essential to the practice of the invention.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

Further, no admission is made that any reference, including any patent or patent document, cited in this specification constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinency of any of the documents cited herein.

### EXAMPLES

### Example 1

### DNA Isolation Efficiency as a Function of Polymer Materials

The effect of polymer composition on DNA extraction efficiency from whole blood was measured using an automated DNA purification instrument (MAXWELL® 16 system, Promega Corp., Madison, Wis.) and a real-time PCR instrument (STRATAGENE MX3005P PCR system, Agilent Technologies, La Jolla, Calif.).

Five sets of six plungers were fabricated from various polymer materials. The plungers were similar in shape to FIG. 1, and approximately 5.4 mm long by 1 mm wide. One set of plungers was fabricated from virgin polypropylene (PP) resin, one set was prepared from polyethylene terephthalate (PET) resin (VALOX®, SABIC Innovative Plastics, Pittsfield, Mass., solubility parameter about 9.9 (cal/cm³)^{1/2}), two sets were prepared from a polyoxymethylene resin (DELRIN®, I.E. du Pont de Nemours, Wilmington, Del., solubility parameter about 11.0-11.1 (cal/cm³)^{1/2}), and one set was prepared from an acrylonitrile butadiene styrene (ABS) resin (TERLUX® 2802HD, BASF, Ludwigshafen, Germany, solubility parameter greater than about 9.5 (cal/cm³)^{1/2}). One set of DELRIN® plungers was unscented, and the second set of DELRIN® plungers had approximately 1% vanilla extract added to the melted DELRIN® prior to being formed into plungers.

A commercially-available DNA isolation protocol and reagent set (DNA IQ™ Casework Sample Kit for MAXWELL® 16, Promega Corp.) was used to isolate the genomic DNA from the whole blood sample and to prepare the isolated DNA for amplification of short tandem repeat (STR) markers. See, Promega Corporation, “DNA Purification,” (Chapter 9) Protocol and Applications Guide (2009), incorporated by reference herein in its entirety. Thirty 500 nL samples of human whole blood were placed in 50 separate reference sample cartridges (DNA IQ™, Promega Corp.) for the MAXWELL® 16 Instrument. The cartridges were constructed of polypropylene. An identically-shaped plunger, prepared from the appropriate polymer material, as described above, was placed in each of the sample cartridges. Accordingly, there were five sets of six separate cartridges with a plunger made from the same polymer, for a total of 30 cartridges. Once the cartridges were prepared with a sample and plunger, the cartridges were placed in the automated DNA purification instrument (MAXWELL® 16), along with recommended elution tubes and buffer, and the automated DNA purification instrument was operated according to the manufacturer's recommendations for the isolation protocol.

At the conclusion of the automated DNA purification cycle, the elution tubes containing the isolated DNA were removed from the instrument. The DNA was then amplified and assayed using a real-time PCR instrument (STRATAGENE MX3005P) using PCR primers specific for STRs along with fluorescent probes to quantify the results (PLEXOR HY™ system, Promega Corp.). Commercially-available PCR assay analysis software (PLEXOR ANALYSIS™, Promega Corp.) was then used to quantify the amount of DNA isolated from each of the samples. The results of the analysis are shown in Table 2.

As an additional control, DNA from five 500 nL samples of the same blood were extracted using manual separation techniques using the reagents and magnetic particles similar to those used in the protocol for automated DNA isolation (above). See, Promega Corporation, “DNA IQ™ Casework Sample Kit for Maxwell® 16” (Technical Bulletin) (2009), incorporated by reference herein in its entirety. All of the manual isolation steps were performed in 1.5 mL polypropylene microcentrifuge tubes. The isolated DNA from the manual extraction was amplified and assayed with a real-time PCR instrument (STRATAGENE MX3005P) as above. The results of the manual isolation are also included in Table 2.

TABLE 2
Y-specific quantitation of human male DNA

| | Amount of DNA isolated from sample (in nanograms) | | | | | | |
|---|---|---|---|---|---|---|---|
| Plunger | Sample # | | | | | | |
| Polymer | 1 | 2 | 3 | 4 | 5 | 6 | Average |
| PP | 9 | 13 | 15 | 11.5 | 17 | 16.5 | 13.7 |
| VALOX ® | 15.5 | 21 | 23 | 17 | 12.5 | 20.5 | 18.3 |
| DELRIN ® | 21.5 | 24.5 | 23.5 | 27 | 37 | 29.5 | 27.2 |
| vanilla | 26.5 | 29 | 26.5 | 29 | 25 | 27.5 | 27.3 |
| DELRIN ® | | | | | | | |
| TERLUX ® | 25 | 28 | 22 | 29.5 | 29 | 26.5 | 26.7 |
| Manual (PP) | 18.5 | 15 | 18 | 20.5 | 25 | | 18.3 |

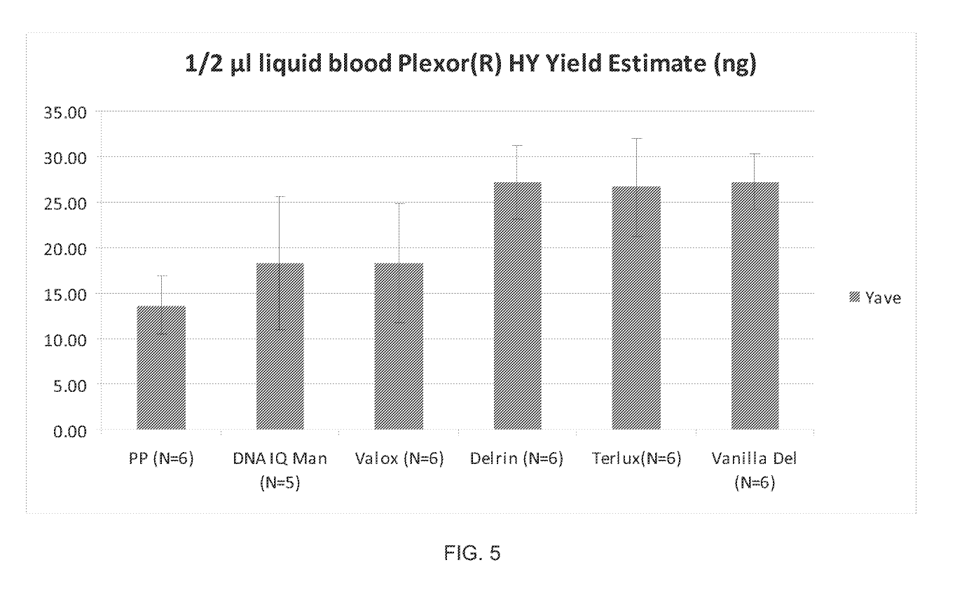

The average isolated DNA from each sample is shown graphically in FIG. 5. The error bars represent one standard deviation for the averaged results. As can be see from FIG. 5, DELRIN® and TERLUX® plungers are almost twice as efficient in extracting DNA as compared to polypropylene.

### Example 2

### DNA Isolation Efficiency Using Nylon 6,6 Polymer Material

The experiments were carried out as described in Example 1, using one set of plungers was fabricated from virgin polypropylene (PP) resin, and one set prepared from Nylon 6,6 resin (solubility parameter about 13.6 (cal/cm³)^{1/2}).

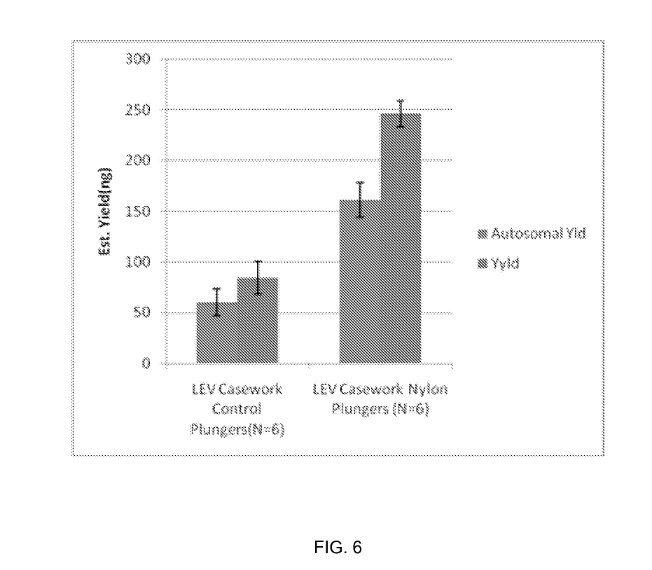

The average isolated DNA from each sample is shown graphically in FIG. 6. The error bars represent one standard deviation for the averaged results. As can be see from FIG. 6, Nylon 6,6 plungers are more than twice as efficient in extracting DNA as compared to polypropylene.

All patents, publications and references cited herein are hereby fully incorporated by reference. In case of conflict between the present disclosure and incorporated patents, publications and references, the present disclosure should control.

1. A process for isolating nucleic acids from a sample, comprising contacting the sample with consumable analytical plasticware comprising a polymer having a solubility parameter of 9.5 (cal/cm³)^{1/2} or greater while isolating the nucleic acids from the sample.

2. The process of claim 1, wherein the polymer has a solubility parameter of about 11.0 (cal/cm³)^{1/2} or greater.

3. The process of claim 1, wherein the plasticware is a plunger, a cartridge, a pipette tip, a sample vial, a tube, a multi-well plate, a dish, a syringe, a spatula, a probe, a swab, a section of tubing, a filter, a filter basket, or a vessel.

4. The process of claim 3, wherein the plasticware is a plunger.

5. The process of claim 3, wherein the polymer comprises polyethylene terephthalate (PET), polybutylene terephthalate (PBT), polyoxymethylene, acrylonitrile butadiene styrene (ABS), vinyl acetate, polyvinyl chloride (PVC), nylon or acrylonitrile.

6. The process of claim 5, wherein the polymer comprises acrylonitrile butadiene styrene (ABS).

7. The process claim of 5, wherein the polymer comprises polyoxymethylene.

8. The process claim of 3, wherein the process additionally comprises contacting the sample with silica or cellulose.

9. The process of claim 8, wherein the process additionally comprises contacting the sample with a silica particle.

10. The process of claim 9, wherein the process additionally comprises contacting the sample with a paramagnetic particle.

11. The process of claim 1, further comprising contacting the sample with consumable analytical plasticware comprising a polymer having a solubility parameter less than 9.5 (cal/cm³)^{1/2} to drive the nucleic acids toward the consumable analytical plasticware comprising a polymer having a solubility parameter of 9.5 (cal/cm³)^{1/2} or greater.

12. Consumable analytical plasticware comprising a polymer having a solubility parameter of about 9.5 (cal/cm³)^{1/2} or greater.

13. The consumable analytical plasticware of claim 12, wherein the plasticware is a plunger, a cartridge, a pipette tip, a sample vial, a tube, a multi-well plate, a dish, a syringe, a spatula, a probe, a swab, a section of tubing, a filter, a filter basket, or a vessel.

14. The consumable analytical plasticware of claim 13, wherein the polymer has a solubility parameter of about 11.0 (cal/cm³)^{1/2} or greater.

15. The consumable analytical plasticware of claim 14, wherein the polymer comprises polyethylene terephthalate (PET), polybutylene terephthalate (PBT), polyoxymethylene, acrylonitrile butadiene styrene (ABS), vinyl acetate, polyvinyl chloride (PVC), nylon, or acrylonitrile.

16. The consumable analytical plasticware of claim 13, wherein the plasticware is a plunger.

17. The consumable analytical plasticware of claim 15, wherein the polymer comprises acrylonitrile butadiene styrene (ABS).

18. The consumable analytical plasticware of claim 15, wherein the polymer comprises polyoxymethylene.
